(19) United States
(12) Patent Application Publication
(10) Pub. No.: US 2018/0256770 A1
(43) Pub. Date: Sep. 13, 2018

(54) Compact Air Washer

(71) Applicants: Dae Kyu Jang, Gyeonggi-do (KR); HATA CO., LTD., Gyeonggi-do (KR)

(72) Inventor: Dae Kyu Jang, Gyeonggi-do (KR)

(21) Appl. No.: 15/759,860

(22) PCT Filed: Sep. 18, 2016
(86) PCT No.: PCT/KR2016/010392

§ 371 Date: Mar. 14, 2018

(30) Foreign Application Priority Data
Sep. 14, 2015 (KR) 10-2015-0129562
Apr. 22, 2016 (KR) 10-2016-0049478

(51) Int. Cl. A61L 9/12; B01D 46/42; A61L 9/22

(52) U.S. Cl. CPC A61L 9/12; A61L 9/22; B01D 46/42

(57) ABSTRACT

The present invention relates to a compact air cleaner which includes: an external case having an air inlet through which air is introduced; an interchangable filter module having a filter for filtering the air introduced through the air inlet and interchangeably arranged inside the external case; a filter guide unit having a plurality of sterilization LEDs for sterilizing bacteria on the filter and arranged within the interchangeable filter module; and a module cap having an air outlet through which the air filtered through the filter is discharged and detachably coupled to one side of the interchangeable filter module.

### TECHNICAL FIELD

The present disclosure relates to a compact air cleaner, and more particularly, to a compact air cleaner which has a compact shape for improving beauty of an outer appearance thereof, may improve efficiency of air cleaning due to an efficient structure for the air cleaning, has a filter that may be more easily replaced as compared to the conventional products, and may extend a replacement period of the filter as well.

### BACKGROUND ART

An air cleaner is collectively referred to as a device configured to clean air by filtering dust, bacteria, or the like in the air. There are various air cleaners according to types of a built-in filter.

Since polluted air may damage health of a resident and degrade quality of life, various kinds of air purifying products, for example, the air cleaner, have been used to prevent this phenomenon.

Among the air cleaner, there is a product equipped with an air cleaning function alone, and a product added to an air conditioner as one function.

In addition, it has been known that there are various air cleaners from a small-sized air cleaner to a large-sized air cleaner according to sizes.

Meanwhile, it has been known that since the air cleaner is in charge of a main function of filtering the dust, the bacteria, and the like in the air as described above, it is most important to clean a filter, particularly, to replace the filter.

In the case of the large-sized air cleaner, since an inner space thereof is large, a structure in which a filter is replaced in a state in which a front case is opened may be sufficiently implemented, which is like an air conditioner. However, in the case of the small-sized air cleaner, it is not easy to implement a structure in which a filter is replaced due to spatial limitation.

Even when it is easy to replace the filter, if a replacement period is too short, inconvenience in use may occur.

As a result, the small-sized air cleaner should have an efficient structure for air cleaning even while having a compact shape for improving beauty of an outer appearance due to product characteristics, and particularly, should have a structure in which even while a filter may be easily replaced, a replacement period may increase. However, until now, since a small-sized air cleaner satisfying these requirements has not been disclosed, there arises a need for the small-sized air cleaner having these requirements.

Furthermore, when such an air cleaner may be equipped with a speaker function, since the air cleaner may additionally provide the speaker function in addition to an original function such as the air cleaning, it is expected to extend functionality. Thus, the applicant has developed a technology for the above-described matters.

### DISCLOSURE

### Technical Problem

The present disclosure provides a compact air cleaner which has a compact shape for improving beauty of an outer appearance thereof, may improve efficiency of air cleaning due to an efficient structure for the air cleaning, has a filter that may be more easily replaced as compared to the conventional products, and may extend a replacement period of the filter as well.

Further, the present disclosure also provides a compact air cleaner which is equipped with a Bluetooth speaker to extend functionality.

### Technical Solution

A compact air cleaner may include an external case having air inlets into which air is introduced, an interchangeable filter module having a filter configured to filter the air introduced through the air inlets and interchangeably arranged inside the external case, a filter guide unit arranged inside the interchangeable filter module and having a plurality of sterilization light emitting diodes (LEDs), and a module cap having air outlets from which the air filtered by the filter is discharged and detachably coupled to one side of the interchangeable filter module.

The interchangeable filter module may further include a filter frame supporting the filter such that the filter is mounted thereon.

Detachable coupling portions detachably coupled to each other may be provided in the module cap and the filter frame.

The filter guide unit may include a unit base, a fan module spaced apart from the unit base to guide the air to the air outlets, and a plurality of wing flanges radially arranged between the unit base and the fan module and having the plurality of sterilization LEDs formed therein.

The compact air cleaner may include a button-type handle coupled to the module cap to elastically move upward/downward.

A detachable scent container case may be detachably coupled to an end of the button-type handle.

The external case may include an air inlet basket having the plurality of air inlets formed along a circumferential direction of the air inlet basket, and a case body having a hollow cylindrical structure, coupled to the air inlet basket, and having at least one switch formed at an end thereof.

The compact air cleaner may further include a Bluetooth speaker provided inside the external case.

The Bluetooth speaker may be arranged below the filter guide unit.

The filter guide unit may include a unit body having a plurality of wing flanges formed therein and coupled to the Bluetooth speaker on one side of a lower portion thereof, a plasma module mounted on the unit body to sterilize bacteria in the filter, a fan module connected to the unit body to guide the air to the air outlets, and a button-type handle connected to the fan module to selectively operate the fan module, and arranged to move upward/downward in a through-hole of the module cap.

The filter guide unit may further include an air freshener container arranged to be adjacent to the plasma module.

The filter guide unit may further include a filter detecting unit provided on one side of the unit body to detect whether the interchangeable filter module exists or not.

The external case may include an outer air inlet basket having a plurality of first air inlets formed along a circumferential direction thereof, an inner air inlet basket having a plurality of second air inlets formed along a circumferential direction thereof to communicate with the plurality of first air inlets, and arranged inside the outer air inlet basket, and a case body having a hollow cylindrical structure and detachably coupled to the outer air inlet basket.

First detachable coupling portions detachably coupled to each other may be provided in the case body and the outer air inlet basket.

The interchangeable filter module may include a filter frame supporting the filter such that the filer is mounted thereon, and second detachable coupling portions detachably coupled to each other may be provided in the module cap and the filter frame.

The Bluetooth speaker may include a cylindrical speaker body, speaker units coupled to opposite ends of the cylindrical speaker body and having Bluetooth communication modules and amplifiers formed in the speaker units, and a sound guide tube arranged at a lower end of the cylindrical speaker body to communicate with the cylindrical speaker body at one point inside the Bluetooth speaker and arranged along a lengthwise direction of the cylindrical speaker body.

The compact air cleaner may further include module protecting and shielding cases arranged radially outward of the interchangeable filter module to protect the interchangeable filter module.

### Advantageous Effects

According to the present disclosure, provided is a compact air cleaner which has a compact shape for improving beauty of an outer appearance thereof, may improve efficiency of air cleaning due to an efficient structure for the air cleaning, has a filter that may be more easily replaced as compared to the conventional products, and may extend a replacement period of the filter as well.

Further, according to the present disclosure, provided is a compact air cleaner which is equipped with a Bluetooth speaker to extend functionality.

### DESCRIPTION OF DRAWINGS

FIG. 1 is a perspective view illustrating a compact air cleaner in a state in which a button-type handle is pushed down according to a first embodiment of the present disclosure;

FIG. 2 is a perspective view illustrating the compact air cleaner in a state in which the button-type handle is pushed up according to the first embodiment of the present disclosure;

FIG. 3 is an exploded perspective view of FIG. 1;

FIG. 4 is a rear perspective view of FIG. 3;

FIG. 5 is an exploded perspective view illustrating a filter guide unit and a module cap of a compact air cleaner according to a second embodiment of the present disclosure;

FIG. 6 is a rear perspective view of FIG. 5;

FIG. 7 is a perspective view illustrating a compact air cleaner according to a third embodiment of the present disclosure;

FIGS. 8 to 11 are views illustrating states in which external configurations are removed from FIG. 7 one by one;

FIG. 12 is an exploded perspective view of FIG. 7;

FIG. 13 is an exploded perspective view illustrating an external case;

FIG. 14A is a perspective view illustrating the filter guide unit;

FIG. 14B is a cross-sectional perspective view illustrating a Bluetooth speaker of FIG. 14A;

FIG. 15 is an exploded perspective view of FIG. 14A; and

FIG. 16 is a side view illustrating a unit body area.

[Reference numerals]

| | |
|---|---|
| 100: Compact air cleaner | 110: External case |
| 120: Air inlet basket | 121: Air inlet |
| 130: Case body | 131: Switches |
| 140: Interchangeable filter | 150: Filter |
| module | |
| 160: Filter frame | 170: Filter guide unit |
| 171: Unit base | 172: Fan module |
| 173: Wing flanges | 175: Sterilization LEDs |
| 180: Module cap | 181: Air outlets |
| 185: Button-type handle | 190: Detachable coupling portions |
| 191: Detachable protrusions | 192: Protrusion accommodating portions |
| 300: Compact air cleaner | 310: External case |
| 321: Outer air inlet basket | 321a: First air inlets |
| 322: Inner air inlet basket | 322a: Second air inlets |
| 330: Case body | 330a: Switch hole |
| 331: Switches | 340: Interchangeable filter module |
| 350: Filter | 360: Filter frame |
| 365a, 365b: Module | 370: Filter guide unit |
| protecting and shielding | |
| cases | |
| 371: Unit body | 372: Wing flanges |
| 373: Plasma module | 374: Fan module |
| 375: Button-type handle | 376: Air freshener container |
| 377: Bluetooth speaker | 378: Filter detecting unit |
| 380: Module cap | 381: Air outlets |
| 382: Through-hole | 391: First detachable coupling portions |
| 391a: First detachable | 391b: First protrusion accommodating |
| protrusions | portions |
| 392: Second detachable | 392a: Second detachable |
| coupling portions | protrusions |

### MODES OF THE INVENTION

Hereinafter, exemplary embodiments of the present disclosure will be described with reference to the accompanying drawings.

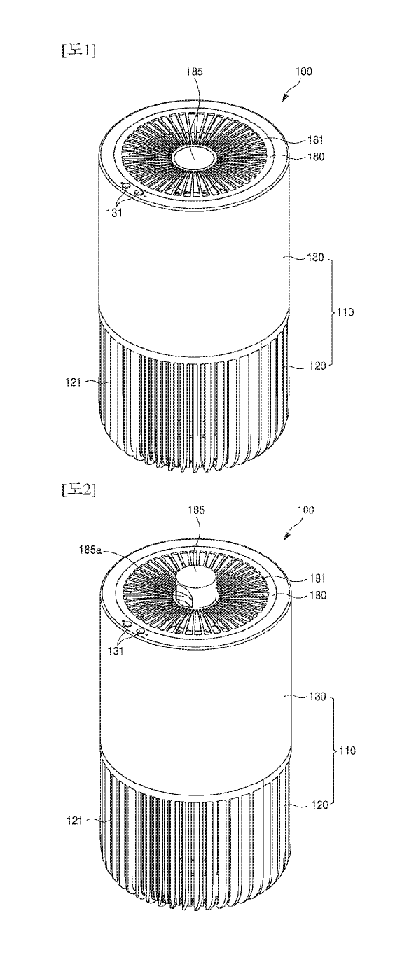

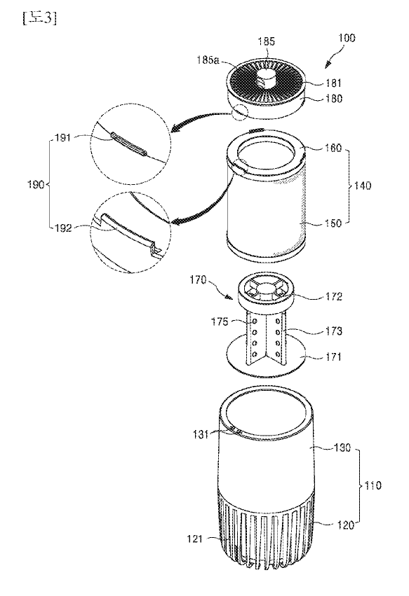

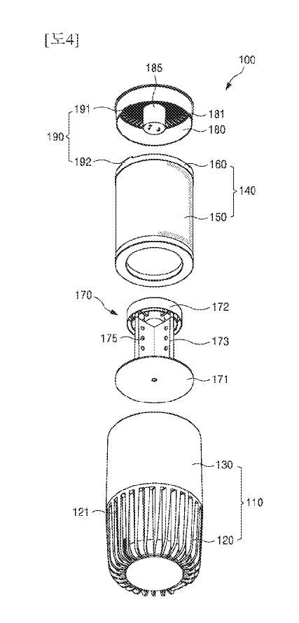

FIG. 1 is a perspective view illustrating a compact air cleaner in a state in which a button-type handle is pushed down according to a first embodiment of the present disclosure, FIG. 2 is a perspective view illustrating the compact air cleaner in a state in which the button-type handle is pushed up according to the first embodiment of the present disclosure, FIG. 3 is an exploded perspective view of FIG. 1, and FIG. 4 is a rear perspective view of FIG. 3.

Referring to the drawings, a compact air cleaner 100 according to the present embodiment has a compact shape for improving beauty of an outer appearance thereof, may improve efficiency of air cleaning due to an efficient structure for the air cleaning, has a filter 150 that may be more easily replaced as compared to the conventional products, and may extend a replacement period of the filter as well. The compact air cleaner 100 may include an external case 110, an interchangeable filter module 140, a filter guide unit 170, and a module cap 180.

First, the external case 110 defines an outer appearance of the compact air cleaner 100 according to the present embodiment, which is like FIGS. 1 and 2.

In the present embodiment, the external case 110 has a generally cylindrical shape. Thus, the external case 110 is sufficient to have the compact shape for improving the beauty of the outer appearance. Also, the external case 110 is good for inducing air flow.

Such an external case 110 includes an air inlet basket 120 and a case body 130.

Although the air inlet basket 120 and the case body 130 may be integrally formed, the air inlet basket 120 and the case body 130 may be separately formed and may be coupled to each other.

The air inlet basket 120 forms the lowermost end of the compact air cleaner 100 according to the present embodiment. Air inlets 121 into which air is introduced are formed in the air inlet basket 120.

The plurality of air inlets 121 are arranged in a slot shape along a circumferential direction of the air inlet basket 120 at equal angular intervals. Thus, the air inlets 121 are effective in introducing the air into the compact air cleaner 100 in various directions.

As described above, the case body 130 has a hollow cylindrical structure, and is coupled to the air inlet basket 120. When the air inlet basket 120 and the case body 130 are separately formed, a coupling scheme may be anything such as a screw scheme, a hook scheme, and a press-fitting scheme.

A plurality of switches 131 for an ON/OFF operation for electric power, setting of a timer, operations of sterilization LEDs 175, and the like are provided on one side of an upper end of the case body 130.

For example, when any one of the plurality of switches 131 is pressed, the sterilization LEDs 175 may be operated.

Next, the interchangeable filter module 140, which is a module interchangeably arranged inside the external case 110, filters the air introduced into the compact air cleaner 100 through the air inlets 121 of the external case 110.

Such an interchangeable filter module 140 may include the filter 150 configured to filter the air introduced through the air inlets 121 and a filter frame 160 supporting the filter 150 such that the filter 150 is mounted thereon.

The filter 150 may be coupled to an outer side of the filter frame 160 in an insertion manner. Thus, after the device is separated as illustrated in FIG. 3, it is easy to replace the filter 150.

Next, the filter guide unit 170 is arranged inside the interchangeable filter module 140, and serves to sterilize bacteria in the filter 150. To this end, the filter guide unit 170 has the plurality of sterilization LEDs 175.

Such a filter guide unit 170 includes a unit base 171, a fan module 172 spaced apart from the unit base 171 to guide the air toward air outlets 181 of the module cap 180, and a plurality of wing flanges 173 radially arranged between the unit base 171 and the fan module 172 and having the plurality of sterilization LEDs 175 provided therein.

Since the plurality of sterilization LEDs 175 are provided in the plurality of wing flanges 173 in plurality, the sterilization LEDs 175 are easy to sterilize the bacteria in the filter 150 by emitting rays that are harmless to a human body.

In the present embodiment, the sterilization LEDs 175 are arranged in the filter guide unit 170 arranged in the interchangeable filter module 140. Further, since such sterilization LEDs 175 sterilize the bacteria in the filter 150, a lifespan and a replacement period of the filter 150 may increase.

Also, a structure in which the filter guide unit 170 rotates may be selectively employed. In this case, sterilization efficiency for the filter 150 may be further doubled.

Finally, the module cap 180 is a member detachably coupled to the filter frame 160 of the interchangeable filter module 140.

The air outlets 181 from which the air filtered by the filter 150 is discharged are formed in such a module cap 180.

Further, a button-type handle 185 is coupled to a central portion of the module cap 180 to elastically move upward/downward.

Although not illustrated in detail, since the button-type handle 185 has a built-in spring, the button-type handle 185 may be pushed down when being pressed once as illustrated in FIG. 1, and may be pushed up when being pressed once again as illustrated in FIG. 2.

Further, when the button-type handle 185 is pushed up as illustrated in FIG. 2, the button-type handle 185 may serve as a handle configured to separate components such as the interchangeable filter module 140 and the filter guide unit 170 from the external case 110.

In this way, in order to allow the button-type handle 185 to serve as the handle, gripping grooves 185a are formed at side portions of the button-type handle 185 to make gripping easy. The gripping grooves 185a may be symmetrically provided on opposite sides of the button-type handle 185.

Meanwhile, as illustrated in FIG. 2, the module cap 180 and the interchangeable filter module 140 should be connected to each other such that the button-type handle 185 is pushed up to lift up the components such as the interchangeable filter module 140 and the filter guide unit 170 from the external case 110.

To this end, detachable coupling portions 190 detachably coupled to each other are provided in the module cap 180 and the filter frame 160.

Various schemes such as a screw scheme or a hook scheme may be applied to the detachable coupling portions 190. In the present embodiment, the detachable coupling portions 190 having detachable protrusions 191 provided at a circumference of the module cap 180 and protrusion accommodating portions 192 which is provided at a circumference of the filter frame 160 and to which the detachable protrusions 191 of the module cap 180 are selectively coupled are applied.

Accordingly, when the module cap 180 rotates in one direction while being in contact with the filter frame 160, the detachable protrusions 191 are fitted in the protrusion accommodating portions 192 so that the module cap 180 and the filter frame 160 may be connected to each other as one body, and in the opposite case, the module cap 180 and the filter frame 160 may be separated from each other.

An effect of the compact air cleaner 100 having the above-described configuration will be described below.

First, after the device is assembled, the device is switched on by operating one of the plurality of switches 131 provided on one side of an upper end of the case body 130.

In this state, after being filtered by the filter 150, the air introduced through the air inlets 121 of the external case 110 may be discharged to the outside through the air outlets 181 of the module cap 180.

At this time, since the sterilization LEDs 175 sterilize the bacteria in the filter 150, a lifespan and a replacement period of the filter 150 may increase.

The compact air cleaner 100 having the above-described structure and effect according to the present embodiment has the compact shape for improving the beauty of the outer appearance thereof, may improve the efficiency of the air cleaning due to the efficient structure for the air cleaning, has the filter 150 that may be more easily replaced as compared to the conventional products, and may extend the replacement period of the filter 150 as well.

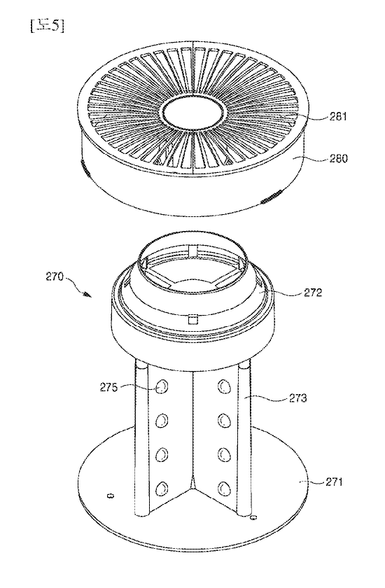

FIG. 5 is an exploded perspective view illustrating a filter guide unit and a module cap of a compact air cleaner according to a second embodiment of the present disclosure, and FIG. 6 is a rear perspective view of FIG. 5.

Referring to the drawings, the compact air cleaner according to the present embodiment may also include a filter guide unit 270 and a module cap 280.

First, the filter guide unit 270 may include a unit base 271, a fan module 272 spaced apart from the unit base 271 to guide the air toward air outlets 281 of the module cap 280, and a plurality of wing flanges 273 radially arranged between the unit base 271 and the fan module 272 and having a plurality of sterilization LEDs 275 provided therein.

In the present embodiment, the shape and the structure of the fan module 272 are different from those according to the above-described embodiment. That is, in the present embodiment, the fan module 272, which does not have a simple ring shape like the above-described embodiment but has a double ring structure, has a shape in which an upper portion thereof is narrower than a lower portion thereof. This structure may advantageously guide and blow a scent emitted through a detachable scent container case 290, which will be described below.

Next, air outlets 281 from which the air filtered by a filter 250 is discharged are formed in the module cap 280, and a button-type handle 285 is coupled to a central portion of the module cap 280 to elastically move upward/downward, which is not different from the above-described embodiment.

However, in the present embodiment, the detachable scent container case 290 is coupled to an end of the button-type handle 285. An air freshener and the like may be accommodated in the detachable scent container case 290.

Accordingly, when being rotated in a forward direction, the detachable scent container case 290 may be coupled to the end of the button-type handle 285, and when being rotated in a reverse direction, the detachable scent container case 290 may be separated from the end of the button-type handle 285. In a state in which the detachable scent container case 290 is separated, when the air freshener and the like is inserted into the detachable scent container case 290 and the detachable scent container case 290 is tightened, a scent generating function as well as an air cleaning function may be achieved. In particular, replacement becomes easy due to the detachable scent container case 290.

Even when the present embodiment is applied, the compact air cleaner 100 has the compact shape for improving the beauty of the outer appearance thereof, may improve the efficiency of the air cleaning due to the efficient structure for the air cleaning, has the filter 150 that may be more easily replaced as compared to the conventional products, and may extend the replacement period of the filter 150 as well.

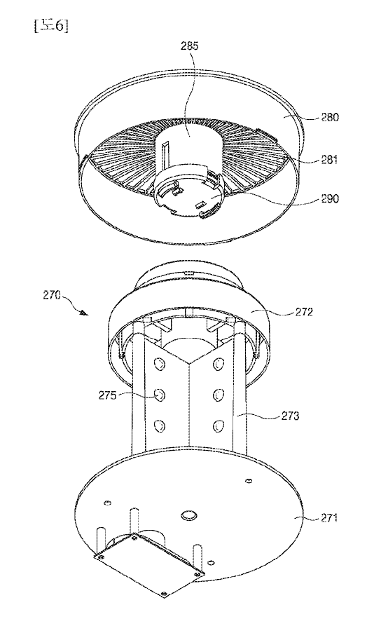

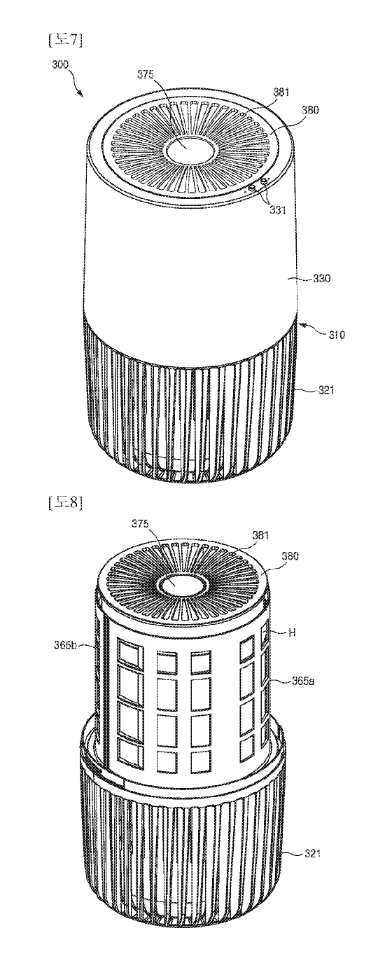

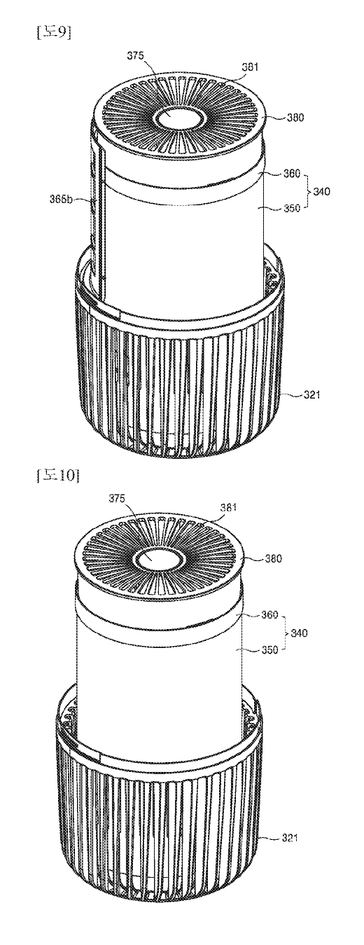

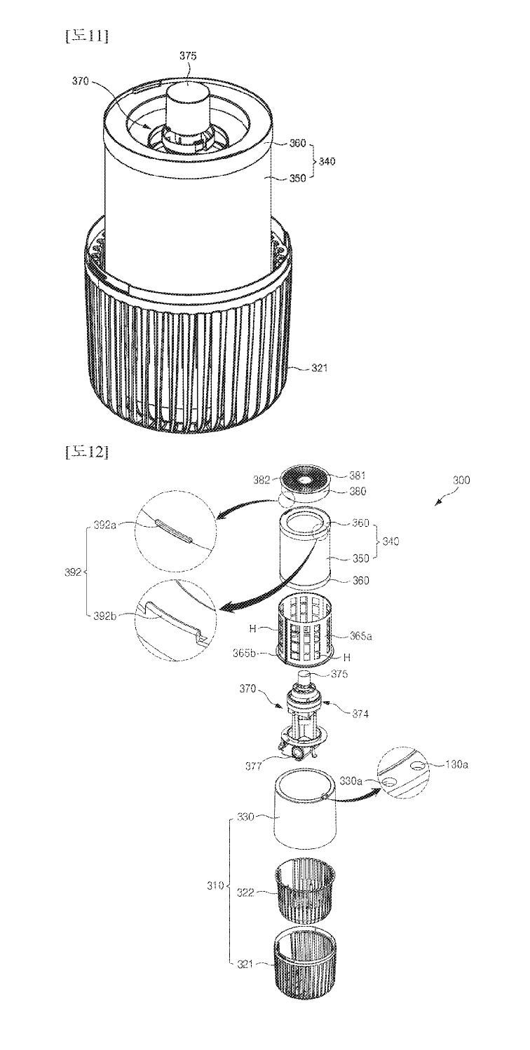

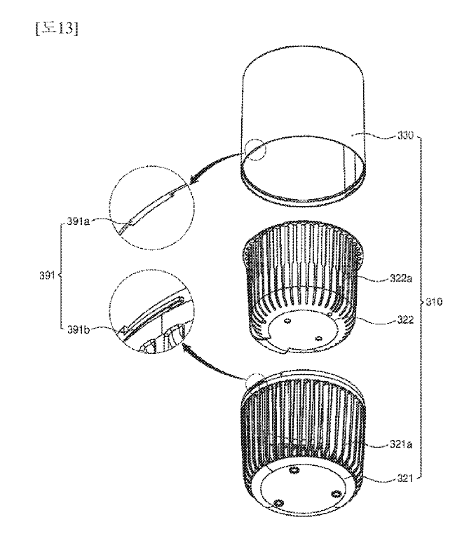

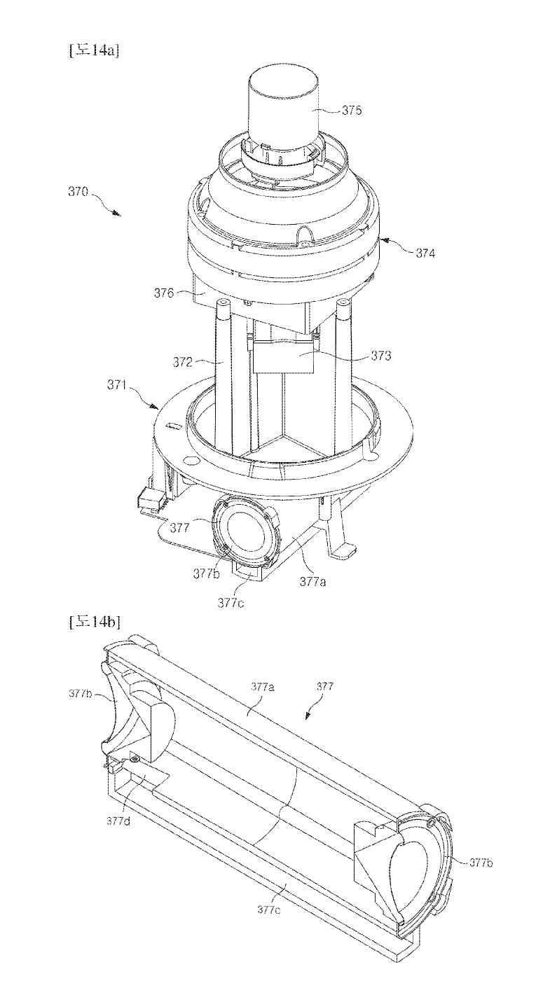

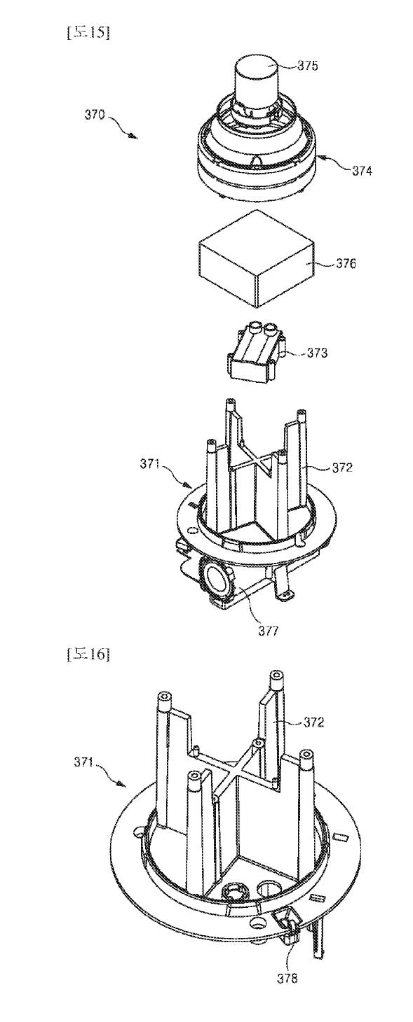

FIGS. 7 to 16 are views illustrating a compact air cleaner according to a third embodiment of the present disclosure. FIG. 7 is a perspective view illustrating a compact air cleaner according to a third embodiment of the present disclosure, FIGS. 8 to 11 are views illustrating states in which external configurations are removed from FIG. 7 one by one, FIG. 12 is an exploded perspective view of FIG. 7, FIG. 13 is an exploded perspective view illustrating an external case, FIG. 14A is a perspective view illustrating the filter guide unit, FIG. 14B is a cross-sectional perspective view illustrating a Bluetooth speaker of FIG. 14A, FIG. 15 is an exploded perspective view of FIG. 14A, and FIG. 16 is a side view illustrating a unit body area.

Referring to the drawings, a compact air cleaner 300 according to the third embodiment of the present disclosure, which may improve efficiency of air cleaning due to an efficient structure, has a filter that may be easily replaced, and has a Bluetooth speaker 377 embedded therein to extend functionality. The compact air cleaner 300 may include an external case 310, an interchangeable filter module 340, module protecting and shielding cases 365a and 365b, a filter guide unit 370, a module cap 380, and the Bluetooth speaker 377.

First, the external case 310 defines an outer appearance of the compact air cleaner 300 according to the present embodiment, which is like the above-described embodiments.

In the present embodiment, the external case 310 may include an outer air inlet basket 321, an inner air inlet basket 322, and a case body 330.

The outer air inlet basket 321 forms the lowermost end of the compact air cleaner 300 according to the present embodiment. First air inlets 321a into which the air is introduced are formed in the outer air inlet basket 321.

The first air inlets 321a are arranged in a slot shape along a circumferential direction of the outer air inlet basket 321 at equal angular intervals. Thus, the first air inlets 321a are effective in introducing the air into the compact air cleaner 300 in various directions.

The inner air inlet basket 322 is a net-shaped structure arranged inside the outer air inlet basket 321. The inner air inlet basket 322 has a similar shape to that of the outer air inlet basket 321. That is, a plurality of second air inlets 322a communicating with the first air inlets 321a of the outer air inlet basket 321 is formed in the inner air inlet basket 322 along a circumferential direction. Thus, external air may be introduced into the device through the first air inlets 321a and the second air inlets 322a.

Although the outer air inlet basket 321 and the inner air inlet basket 322 are separated from each other in the present embodiment, the outer air inlet basket 321 and the inner air inlet basket 322 may be formed integrally.

The case body 330 is a part having a hollow cylindrical shape and detachably coupled to the outer air inlet basket 321.

That is, in the present embodiment, the case body 330 has a hollow cylindrical shape and is coupled to the outer air inlet basket 321. First detachable coupling portions 391 detachably coupled to each other are provided in the case body 330 and the outer air inlet basket 321 such that the case body 330 and the outer air inlet basket 321 may be detachably coupled to each other.

Various schemes such as a screw scheme and a hook scheme may be applied to the first detachable coupling portions 391. In the present embodiment, as illustrated in FIG. 13, applied is a hook coupling scheme using first detachable protrusions 391a which are provided at a circumference of the case body 330 and first protrusion accommodating portions 391b which are provided at a circumference of the outer air inlet basket 321 and to which the first detachable protrusions 391a of the case body 330 are selectively coupled. Of course, an opposite structure may be applied in which the first protrusion accommodating portions 391b are provided in the case body 330 and the first detachable protrusions 391a are provided in the outer air inlet basket 321.

Accordingly, when the case body 330 rotates in one direction while being in contact with the outer air inlet basket 321, the first detachable protrusions 391a are fitted in the first protrusion accommodating portions 391b so that the case body 330 and the outer air inlet basket 321 may be connected to each other as one body, and in the opposite case, the case body 330 and the outer air inlet basket 321 may be separated from each other.

Switch holes 330a in which a plurality of switches 331 for an ON/OFF operation of electric power, setting of a timer, and the like is fitted to be exposed to the outside are formed on one side of an upper end of the case body 330.

Next, the interchangeable filter module 340, which is a module interchangeably arranged inside the external case 310, filters the air introduced into the compact air cleaner 300 through the first and second air inlets 321a and 322a of the external case 310.

Such an interchangeable filter module 340 may include a filter 350 configured to filter the air introduced through the first and second air inlets 321a and 322a and a filter frame 360 supporting the filter 350 such that the filter 350 is mounted thereon. The filter 350 may be coupled to an outer side of the filter frame 360 in an insertion manner. Thus, it is easy to replace the filter 350.

Next, the module protecting and shielding cases 365a and 365b serve to protect the interchangeable filter module 340 and shield an inside of the compact air cleaner 300. Thus, even in a state in which the case body 330 is removed to remove the interchangeable filter module 340, and the interchangeable filter module 340 is withdrawn, the inside of the compact air cleaner 300 may be adequately shielded by the net-structured module protecting and shielding cases 365a and 365b. Such module protecting and shielding cases 365a and 365b are arranged radially outward of the interchangeable filter module 340.

Although the module protecting and shielding cases 365a and 365b may be arranged in divided one pair and may be assembled with each other at a corresponding position in the present embodiment, the present disclosure is not limited to the above-described structure. A plurality of ventilation holes H is formed in the module protecting and shielding cases 365a and 365b.

Next, the filter guide unit 370 has a fan module 374 provided at an upper portion thereof, and is arranged inside the interchangeable filter module 340 to serve to insert and withdrawn the interchangeable filter module 340.

Such a filter guide unit 370 may include a unit body 371, a plasma module 373, an air freshener container 376, the fan module 374, and a button-type handle 375.

The unit body 371 is a part constituting an outer appearance of the filter guide unit 370. A plurality of wing flanges 372 is coupled to the unit body 371,

The plasma module 373 is mounted on the unit body 371 and serves to sterilize bacteria in the filter 350 by generating plasma.

As in the present embodiment, the plasma module 373 is arranged in the filter guide unit 370 arranged in the interchangeable filter module 340. Further, since the plasma module 373 sterilizes the bacteria in the filter 350, a lifespan and a replacement period of the filter 350 may increase.

The air freshener container 376 is arranged to be adjacent to the plasma module 373. Since an air freshener may be diffused from the air freshener container 376 to the outside, fragrant scent in addition to an air cleaning effect may be provided.

The fan module 374 serves to suction the air to guide the air toward air outlets 381 of the module cap 380.

The button-type handle 375 corresponds to a kind of switch which is connected to the fan module 374 to selectively operate the fan module 374 and is arranged in a through-hole 382 of the module cap 380 to move upward/downward. When the button-type handle 375 is pressed, the fan module 374 may be operated, and in the opposite case, the operation of the fan module 374 may be terminated.

Meanwhile, a filter detecting unit 378 is further provided in the filter guide unit 370 according to the present embodiment.

The filter detecting unit 378 may be provided on one side of the unit body 371 constituting the filter guide unit 370, and serves to detect whether the interchangeable filter module 340 is inserted or not. In a state in which the interchangeable filter module 340 is not located in an original position, a control signal of notifying the outside of a signal for the state or preventing the operation of the device from proceeding may be provided

Next, the module cap 380 is a member detachably coupled to the filter frame 360 of the interchangeable filter module 340.

The air outlets 381 from which the air filtered by the filter 350 is discharged are formed in such a module cap 380.

To detachably couple the module cap 380 and the filter frame 360 to each other, second detachable coupling portions 392 detachably coupled to each other are provided in the module cap 380 and the filter frame 360.

Various schemes such as a screw scheme and a hook scheme may be applied to the second detachable coupling portions 392. In the present embodiment, applied is a hook coupling scheme using second detachable protrusions 392a which are provided at a circumference of the module cap 380 and second protrusion accommodating portions 392b which are provided at a circumference of the filter frame 360 and to which the second detachable protrusions 392a of the module cap 380 are selectively coupled. Of course, an opposite structure may be applied in which the second protrusion accommodating portions 392b are provided in the module cap 380 and the second detachable protrusions 392a are provided in the filter frame 360. In the present embodiment, the second protrusion accommodating portions 392b are provided even in the filter frame 360, and thus even when the interchangeable filter module 340 is turned upside down and is coupled to the module cap 380, the interchangeable filter module 340 and the module cap 380 may be detachably coupled to each other using the same structure, so that user's convenience may be improved.

Accordingly, when the module cap 380 rotates in one direction while being in contact with the filter frame 360, the second detachable protrusions 392a are fitted in the second protrusion accommodating portions 392b so that the module cap 380 and the filter frame 360 may be connected to each other as one body, and in the opposite case, the module cap 380 and the filter frame 360 may be separated from each other.

Finally, the Bluetooth speaker 377 is coupled to the filter guide unit 370 inside the external case 310. In particular, the Bluetooth speaker 377 may be coupled to a lower portion of the unit body 371.

The Bluetooth speaker 377 may be formed to have a structure illustrated in FIG. 14B. Speaker units 377b are coupled to opposite ends of a cylindrical speaker body 377a. Bluetooth communication modules (not illustrated) and amplifiers (not illustrated) are provided in the speaker units 377b. A sound guide tube 377c communicating with the cylindrical speaker body 377a through a communication unit 377d at one point inside the Bluetooth speaker 377 and arranged along a lengthwise direction of the cylindrical speaker body 377a may be further provided at a lower end of the cylindrical speaker body 377a. The sound guide tube 377c has one closed end and the other opened end.

In this way, in a structure of the Bluetooth speaker 377, a part of sound generated by the speaker units 377b at the opposite ends is collected inside the cylindrical speaker body 377a and is discharged toward one side through the sound guide tube 377c at once, so that quality of the sound which is softer and has echoes may be formed while the volume of the sound increases.

As in the present embodiment, when the Bluetooth speaker 377 is applied, since a user may listen to music by simply connecting an electronic device such as a smart phone and the Bluetooth speaker 377 to each other in addition to an original function such as the air cleaning, functionality may be extended.

An effect of the compact air cleaner 300 having the above-described configuration according to the third embodiment will be described below.

First, after the device is assembled, the device is switched on by operating one of the plurality of switches 331 arranged on one side of an upper end of the case body 330.

Next, if the fan module 374 of the filter guide unit 370 is operated when the button-type handle 375 is pushed down, the air is introduced through the first and second air inlets 321a and 322a. After being filtered by the filter 350, the introduced air may be discharged to the outside through the air outlets 381 of the module cap 380.

At this time, since the plasma module 373 sterilizes the bacteria in the filter 350, a lifespan and a replacement period of the filter 350 may increase.

Also, when it is necessary to replace the filter 350, the filter 350 may be simply replaced by separating the case body 330, separating the module cap 380, withdrawing the interchangeable filter module 340, replacing the filter 350, and then inserting the above components. At this time, the inside of the compact air cleaner 300 may be properly shielded by the module protecting and shielding cases 365a and 365b.

Meanwhile, when the user wants to use the Bluetooth speaker 377 during the above operation, the user may listen to music by wirelessly connecting the Bluetooth speaker 377 to an audio device or the smart phone.

According to the compact air cleaner 300 having the above-described structure and effect according to the present embodiment, efficiency of air cleaning may be improved due to an efficient structure, a filter may be easily replaced, and the Bluetooth speaker 377 is embedded as well so that functionality may be extended.

In this way, the present disclosure is not limited to the above-described embodiments, and it is obvious to those skilled in the art that various modifications and variations may be conceived without departing from the spirit and the scope of the present invention. Thus, the modifications and the variations belong to the appended claims of the present disclosure.

1. A compact air cleaner comprising:
an external case having air inlets into which air is introduced;
an interchangeable filter module having a filter configured to filter the air introduced through the air inlets and interchangeably arranged inside the external case;
a filter guide unit arranged inside the interchangeable filter module; and
a module cap having air outlets from which the air filtered by the filter is discharged and detachably coupled to the interchangeable filter module.

2. The compact air cleaner of claim 1, wherein the interchangeable filter module further includes,
a filter frame supporting the filter such that the filter is mounted on the filter frame.

3. The compact air cleaner of claim 2, wherein detachable coupling portions detachably coupled to each other are provided in the module cap and the filter frame.

4. The compact air cleaner of claim 1, wherein the filter guide unit includes:
a unit base;
a fan module spaced apart from the unit base to guide the air to the air outlets; and
a plurality of wing flanges radially arranged between the unit base and the fan module.

5. The compact air cleaner of claim 1, further comprising:
a button-type handle coupled to the module cap to elastically move upward/downward.

6. The compact air cleaner of claim 5, wherein a detachable scent container case is detachably coupled to an end of the button-type handle.

7. The compact air cleaner of claim 1, wherein the external case includes:
an air inlet basket having the plurality of air inlets formed along a circumferential direction of the air inlet basket; and
a case body having a hollow cylindrical structure, coupled to the air inlet basket, and having at least one switch formed at an end of the case body.

8. The compact air cleaner of claim 1, further comprising:
a Bluetooth speaker provided inside the external case.

9. The compact air cleaner of claim 8, wherein the Bluetooth speaker is arranged below the filter guide unit.

10. The compact air cleaner of claim 9, wherein the filter guide unit includes:
a unit body having a plurality of wing flanges formed in the unit body and coupled to the Bluetooth speaker on one side of a lower portion of the unit body;
a plasma module mounted on the unit boy to sterilize bacteria in the filter;
a fan module connected to the unit body to guide the air to the air outlets; and
a button-type handle connected to the fan module to selectively operate the fan module, and arranged in a through-hole of the module cap to move upward/downward.

11. The compact air cleaner of claim 10, wherein the filter guide unit further includes:
an air freshener container arranged to be adjacent to the plasma module.

12. The compact air cleaner of claim 10, wherein the filter guide unit further includes,
a filter detecting unit provided on one side of the unit body to detect whether the interchangeable filter module exists or not.

13. The compact air cleaner of claim 8, wherein the external case includes:
an outer air inlet basket having a plurality of first air inlets formed along a circumferential direction of the outer air inlet basket;
an inner air inlet basket having a plurality of second air inlets formed along a circumferential direction of the inner air inlet basket to communicate with the plurality of first air inlets, and arranged inside the outer air inlet basket; and
a case body having a hollow cylindrical structure and detachably coupled to the outer air inlet basket.

14. The compact air cleaner of claim 13, wherein first detachable coupling portions detachably coupled to each other are provided in the case body and the outer air inlet basket.

15. The compact air cleaner of claim 8, wherein the interchangeable filter module includes,
a filter frame supporting the filter such that the filer is mounted on the filter frame, and
wherein second detachable coupling portions detachably coupled to each other are provided in the module cap and the filter frame.

16. The compact air cleaner of claim 8, wherein the Bluetooth speaker includes:
a cylindrical speaker body;
speaker units coupled to opposite ends of the cylindrical speaker body and having Bluetooth communication modules and amplifiers formed in the speaker units; and
a sound guide tube arranged at a lower end of the cylindrical speaker body to communicate with the cylindrical speaker body at one point inside the Bluetooth speaker and arranged along a lengthwise direction of the cylindrical speaker body.

17. The compact air cleaner of claim 8, further comprising:
module protecting and shielding cases arranged radially outward of the interchangeable filter module to protect the interchangeable filter module.
